(19) United States
(12) Patent Application Publication
(10) Pub. No.: US 2003/0082151 A1
(43) Pub. Date: May 1, 2003

(54) EXTENSION OF THE EXPRESSION OF TRANSGENIC PROTEINS BY IMMUNOMODULATION

(76) Inventors: GERHARD SEEMANN, MARBURG-ELNHAUSEN (DE); GUNTER CICHON, BERLIN (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 09/381,344

(22) PCT Filed: Mar. 12, 1998
(86) PCT No.: PCT/EP98/01438

(30) Foreign Application Priority Data
Mar. 21, 1997 (DE) 19711800.3

(51) Int. Cl. A61K 48/00; A61K 39/395; A61K 31/573; A61K 31/525; A61K 31/44; A61K 31/42; A61K 31/277

(52) U.S. Cl. 424/93.21; 424/144.1; 514/251; 514/179; 514/378; 514/352; 514/521

(57) ABSTRACT

The invention relates to the use of one or more immunosuppressants for the production of a pharmaceutical for increasing the tolerance of a mammal to transgenic cells, and to a process for identifying immunosuppressants suitable for this. By the use of pharmaceuticals of this type, the production of the transgenic expression product is markedly prolonged even after discontinuing the immunosuppressant treatment.

The invention relates to the use of one or more immunosuppressants for the production of a pharmaceutical in order to increase the tolerance of a mammal to transgenic cells, and to a process for identifying immunosuppressants suitable for this. By means of the use of pharmaceuticals of this type, the production of the transgenic expression product is markedly prolonged, even after discontinuing the immunosuppressant treatment.

It is known that the insertion of genetic material into cells of a mammal, of man or even of various animals such as horses, sheep, cows, goats, pigs, dogs, mice or rats frequently produces an immunological reaction in vivo. Thus the transgenic cells are killed, for example, by cytotoxic immune cells and thus the expression of one or more proteins or peptides effected by means of the inserted genetic material is ended by this cellular immune reaction. A humoral immune reaction in turn makes difficult or prevents the renewed insertion of the genetic material, e.g. by the neutralization of the vectors by means of specific antibodies (Tripathy S. K. et al., nat Med:2(5), 1996, pages 545-550; Jang J. et al., Gene Ther. 3(2): 1996, pages 137-144).

In order to suppress or to prevent an undesired immune reaction in gene therapy which generates transgenic cells, immunosuppressant substances have therefore been employed in the course of concomitant therapy. A disadvantage, however, proved to be the unpleasant side effects in some cases associated therewith and the increased susceptibility to infection of immunosuppressed organisms, which counteract a longer administration of immunosuppressants (Lockmuller et al. Gene Ther. 3(8), 1996, pages 706-716).

In WO 96/12406, it was described that the humoral immune response to an adenovirus vector can be suppressed by an immunosuppressant concomitant therapy and that repeated vector administration is made possible by this means. In addition to the use of steroids such as dexamethasone, cyclosporin A and 1-amino-19-guanidine-11-hydroxy-4,9,12-triazanondecane-10,13-dione trihydrochloride (deoxyspergualin; called DSG in the following) are especially recommended for this purpose.

The object of the invention is the identification of substances which for a long time afterward—i.e. even after discontinuing the immunosuppressant concomitant therapy—prevent the rapid destruction of the transgenic cells and thus increase the tolerance of a mammal to transgenic cells. Thus, the expression of the transgenic product(s) is maintained longer in vivo. As a consequence, repeated administration of the genetic material could be stopped or at least the frequency could be restricted.

It has been found that only certain immunosuppressants have the desired efficacy. For example, FK 506 and cyclosporin A, which is particularly recommended in WO 96/12406, were unsuitable.

The object is therefore achieved according to the invention by a process for identifying a suitable immunosuppressant or a combination of a number of immunosuppressants which increase the tolerance of a mammal to transgenic cells, and to their use for the production of a pharmaceutical or of a pharmaceutical combination for increasing the tolerance of a mammal, in particular of man, to transgenic cells.

The invention comprises the use of an immunosuppressant or a combination of a number of immunosuppressants for the production of a pharmaceutical or of a pharmaceutical combination for increasing the tolerance of a mammal, in particular of man, to transgenic cells. By this, it is meant that the immunosuppressant(s) can be administered in the form of a single pharmaceutical or in the form of a number of pharmaceuticals which, in turn, can each contain one or more immunosuppressants.

Immunosuppressants are substances which are able to suppress or attenuate immunological reactions. They are used especially in transplantation medicine and for the treatment of autoimmune diseases. Suitable immunosuppressants are, for example, N-(4-trifluoromethylphenyl-5-methylisoxazole-4-carboxamide (leflunomide), DSG, anti-T-cell antibodies, corticosteorids, azathioprine, cyclophosphamide or methotrexate.

The chemical structure of the immunosuppressant—seen from the general concept of the invention—is unimportant, since the decisive feature is found in its action of increasing the tolerance to transgenic cells.

Tolerance to transgenic cells is understood as meaning that the transgenic cells are able, after completion of the immunosuppressant concomitant therapy according to the invention, to produce the transgenic expression products in vivo for longer than they are able to in mammals of a nonimmunosuppressed control group.

Transgenic cells are cells of any type, i.e. animal, vegetable or bacterial cells, preferably cells from mammals, very particularly preferably human cells, into which genetic material has been inserted.

The aim associated with the production of the transgenic cells, for example a gene therapy treatment or else the use of an animal with transgenic cells as an active compound producer, is unimportant for the general concept of the invention. In addition, neither the production process of the transgenic cells nor the genetic material per se are of decisive importance for the invention. The included genetic material can consist, for example, of one or more deoxyribonucleic acid and/or ribonucleic acid chains. The nucleic acid chains can contain promoters, various other gene-regulatory control functions, sequences for specific incorporation into the cellular genome and/or gene sequences which code for specific proteins or peptides. In relation to the cellular genome, the inserted genetic material can be of a foreign or of the same species or even originate from the same individual. A combination thereof is also possible. The specific transgene expression product(s) can be unnatural or natural, for example human, animal, vegetable, bacterial or viral proteins or peptides. Examples are enzymes, receptors, messengers, hormones, growth factors, clotting factors, apolipoproteins, factors affecting the metabolism or cell division, antiinflammatory factors, inhibitors or activators of the cell cycle and of intracellular signal chains, tumor suppressors, elements of the cytoskeleton or of the collective tissue, antigens of disease pathogens or parasites and tumor-associated antigens. Further examples are insulin; hirudin; factor VII; factor IX; factor XIII; von Willebrand factor; antibodies; erythropoietin; human growth hormone; growth factors such as EGF, TGFα, TGFβ, GM-CSF, PDGF, nerve growth factor and others; tumor necrosis factor; interferons; interleukins; p53; tumor therapy; hepatitis virus antigens; HIV antigens, herpesvirus antigens; Borrelia antigens; plasmodium antigens; Trypanosoma antigens, Taenia antigens or human β-glucuronidase.

By the use according to the invention of the immunosuppressant(s), an inhibition of the cellular response to the transgenic cells is in particular achieved. The invention is further distinguished in that approximately 15 days after discontinuing the immunosuppressant pharmaceutical or the pharmaceutical combination, still more than 1.5 times as much, preferably more than twice as much, very particularly preferably more than 5 times as much transgenic expression product can be produced in the treated mammals as in mammals of a nonimmunosuppressed control group. The detection of the expressed transgenic product does not have to take place on the 15th day; it is also possible to carry out measurements before or after this time. It is crucial that just a few days, for example 10 or 20 days, after discontinuing the immunosuppressant treatment, the transgenic expression product is not being produced only in the same or a smaller amount than in nonimmunosuppressed organisms of a control group.

The use according to the invention additionally comprises an administration of the pharmaceutical or the pharmaceutical combination before, during and/or after the administration of the transgenic cells produced in vivo or their production in vivo. Likewise included is the use according to the invention for assisting a gene therapy treatment producing transgenic cells.

In the gene therapeutic treatment to be assisted, genetic material is inserted into cells in vitro or in vivo in the form of one or more nucleic acid chains. This is carried out, for example, with the aid of viral vectors, such as adenoviruses, retro viruses or herpesviruses, or other methods, for example by means of transfection, by direct injection, by gene gun, or with the aid of liposomes, virosomes or receptor-mediated transport systems.

Using the gene therapy to be assisted, disorders can be treated in which a protein or peptide is not produced, or is produced inadequately or only defectively in the body of mammal, but it is also used in order to produce vaccine protection against various disease pathogens or against abnormal endogenous cells as well as for the production of unnatural proteins or peptides in body cells which have a promoting or inhibitory influence on the activities of enzyme or signal cascades or for the production of enzymes which activate specific prodrugs. The gene therapy to be assisted can thus be employed for the treatment of hereditary disorders such as cystic fibrosis, hemophilia, familial hypercholesterole emia, sickle cell anemia, phenylketonuria, of metabolic disorders, such as diabetes; of inflammations; of nerve and brain disorders such as Parkinson's, Alzheimer's or Kreuzfeld-Jakop syndrome, of rheumatic disorders, osteoarthritis, osteoporosis or arthrosis, of carcinomatous disorders; of infectious disorders such as, for example, AIDS or hepatitis or of hormone and growth disorders. The production of vaccine protection against disease pathogens such as viruses, bacteria, fungi, mono- and multicellular parasites and also against abnormal endogenous cells, for example tumor cells, is a further area of gene therapy use to be assisted.

The invention furthermore includes a process for the production of a pharmaceutical or of a pharmaceutical combination in order to increase the tolerance of a mammal to transgenic cells, which comprises bringing an immunosuppressant or a combination of a number of immunosuppressants into a suitable administration form using a physiologically acceptable vehicle and further suitable active compounds, additives or auxiliaries. For example, leflunomide and/or its physiologically tolerable and pharmacologically active salts, derivatives, isomers and metabolites can be used as an immunosuppressant—on its own or also in combination with other immunosuppressants.

The pharmaceutical according to the invention or the pharmaceutical combination according to the invention can be administered, for example, orally, intravenously, subcutaneously, intraperitoneally, percutaneously, cutaneously, topically, by inhalation, intramuscularly, intrathecally, intraocularly, ocularly, buccally, nasally or rectally, preferably intravenously or orally,

A particularly preferred embodiment of the invention relates to the use according to the invention of a combination of a formula (I) or (II)

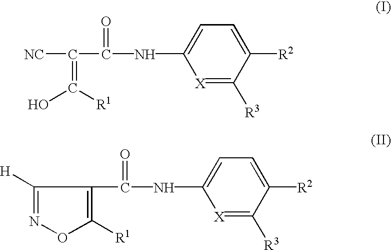

and/or an optionally stereoisomeric form of the compound of the formula I or I, and/or a physiologically tolerable salt of the compound of the formula I,

where

R¹ is

a) (C₁-C₄)-alkyl,

b) (C₃-C₅)-cycloalkyl,

c) (C₂-C₆)-alkenyl or

d) (C₂-C₆)-alkynyl,

R² is

a) —CF₃,

b) —O—CF₃,

c) —S—CF₃,

d) —OH,

e) —NO₂,

f) halogen,

g) benzyl,

h) phenyl,

i) —O-phenyl,

k) —CN or

l) —O-phenyl, mono- or polysubstituted by

1) (C₁-C₄)-alkyl,

2) halogen,

3) —O—CF₃ or

4) —O—CH₃,

R³ is

a) (C₁-C₄)-alkyl,

b) halogen, or

c) a hydrogen atom, and

X is

a) a —CH group or

b) a nitrogen atom,

for the production of the pharmaceutical or of the pharmaceutical combination on its own or also in combination with other pharmacologically active substances, in particular other immunosuppressants.

A compound of the formula I and/or II and/or an optionally stereoisomeric form of the compound of the formula I or II and/or a salt of the compound of the formula I is preferred, where

R¹ is

a) methyl,

b) cyclopropyl or

c) (C₃-C₅)-alkynyl,

R² is —CF₃ or —CN,

R³ is hydrogen atom or methyl, and

X is a —CH group.

Particularly preferably, N-(4-trifluoromethylphenyl)-5-methylisoxazole-4-carboxamide, N-(4-trifluoromethylphenyl)-2-cyano-3-hydroxycrotonamide, 2-cyano-3-cyclopropyl-3-hydroxyacrylic acid (4-cyanophenyl)amide or N-(4-trifluoromethylphenyl)-2-cyano-3-hydroxyhept-2-en-6-ynecarboxamide are employed.

These compounds of the formulae I and/or II or their physiologically tolerable and pharmacologically active salts or derivatives are administered, for example, in a dose of 0.1 to 100 mg/kg, preferably 0.1 to 50 mg/kg, very particularly preferably 0.5 to 20 mg/kg.

The dosage to be used, of course, is dependent on various factors such as the living being to be treated (for example man or animal), the age, weight, general state of health, the degree of severity of the symptoms, the disorder to be treated, possible concomitant disorders, (if present) the nature of the concomitant treatment with other pharmaceuticals, or the frequency of the treatment. In general, the dosages are administered a number of times per day and preferably once to three times per day. The amounts of individual active compound used are based here on the potency of the compound of the formula I or II employed.

The suitable therapy with the compounds of the formula I or II according to the invention thus consists, for example, in the administration of one, two or 3 individual doses of N-(4-trifluoromethylphenyl)-5-methylisoxazole-4-carboxamide in an amount of 2 to 20 mg, preferably 10 or 20 mg.

The invention further relates to a process for identifying a suitable immunosuppressant or a combination of a number of immunosuppressants which increase the tolerance of a mammal to transgenic cells, which comprises measuring the amount of transgenic expression product one or more times after 15, 30, 50 or more days after discontinuing the immunosuppressant treatment, relating it with respect to the amount of transgenic expression product which was produced by nonimmunosuppressed mammals from a corresponding control group and as selection criterion a quantitative factor (=amount of transgenic expression product produced by the immunosuppressant animal group/amount of transgenic expression product produced by the nonimmunosuppressed control animal group; in each case a comparable time reference point) based on, for example, of more than 1.5, or greater than 2, preferably greater than 5, very particularly preferably greater than 10. The detection of the expressed transgenic product does not have to take place only from the 20th day after discontinuing the immunosuppressant treatment. Continuous monitoring of the expression rate of the transgenic product, for example from the time of the production of the transgenic cells, from the 1st day after discontinuing the immunosuppressant treatment or from the 10th day after discontinuing the immunosuppressant treatment, are perfectly possible within the meaning of the invention.

The following examples serve to illustrate the invention. They are not to be understood as a restriction of any type.

### EXAMPLE 1

Cyclosporin A (50-100 mg/kg/day) and DSG (10 mg/kg/day) was administered to the mice over a period of 21 days after administration of a recombinant adenovirus (reporter gene β-galactosidase; Bett et al. Proc. Natl. Acad. Sci. USA, 91 (1994), pages 8802-8806).

The control group reaches a maximum of the reporter gene expression in the liver on the 6th day, the expression is then continuously reduced by the activity of cytotoxic T cells and reaches the starting level after approximately 21 days.

Both in the cyclosporin A and in the DSG group, the expression continues undecreased over the treatment period of 21 days.

In the control group, it was possible to detect the formation of antibodies even 5 days after vector administration. In the cyclosporin A and the DSG group, antibody production was completely eliminated in the doses indicated above.

After discontinuing the immunosuppressants, the reporter gene expression in the cyclosporin A group fell to the starting level over a period of 21 days (42 days after vector administration) while in the DSG group a massive gene expression was still detectable.

In approximately 50% of the animals which were treated with DSG only for 5 days after vector administration, it was still possible to measure approximately 10% of the maximal expression on the 42nd day after vector administration. Obviously, DSG inhibits particularly the early phase of T-cell stimulation very effectively.

### EXAMPLE 2

On day 0, all mice were administered a dose of 1×10 adenoviruses via the tail vein. The recombinant adenoviruses carry the gene for human alpha 1-antitrypsin as a reporter gene found in the serum. This gene has proven to be a practicable reporter, since it is not antigenic in the mouse, but nevertheless can be differentiated from murine alpha 1-antitrypsin using a specific antibody. The half-life of the antiprotease is approximately 3-4 days, so that the serum level reflects the current synthesis capacity of the liver very well.

The total collective was subdivided into five groups of 4 animals each, which were treated as follows:

1st group: control group—no immunosuppressant treatment, administration of saline solution i.p. for the period of 5 days after virus administration.

2nd. group: FK 506 group—administration of 1 mg/kg/day of FK 506 i.p. beginning 1 day before virus administration and then for a period of 5 days.

3rd group: cyclosporin A group—administration of 20 mg/kg/day i.p. beginning 1 day before virus administration and then for a period of 5 days.

4th group: DSG group—administration of 10 mg/kg/day i.p. beginning 1 day before virus administration and then for the period of 5 days.

5th group: DSG++ group—administration of 10 mg/kg/day i.p. beginning 1 day before virus administration and then daily for the period of five days and then twice weekly for the period of 150 days.

NMRI mice were used, since these mice, due to their genetic heterogeneity, develop an immunological reaction pattern which approaches that of a human population more closely than pure inbred strains; the price is an increased variability in the immunological reactivity. In spite of this, the individual groups develop significant reaction patterns.

Table 1 shows the average values of the serum level of human α₁-antitrypsin.

TABLE 1

| Days | FK 506 | Cyclosporin A | Control | DSG | DSG++ |
|---|---|---|---|---|---|
| 5 | 1725000 | 1518750 | 1453750 | 2775000 | 2775000 |
| 10 | 1278750 | 660000 | 616250 | 1560000 | 677500 |
| 20 | 376250 | 264875 | 1055000 | 1826250 | 1178750 |
| 30 | 17475 | 77787 | 117812 | 1001250 | 1082500 |
| 60 | 4258 | 23875 | 37625 | 213500 | 297500 |
| 100 | 313 | 1876 | 12475 | 51750 | 68750 |
| 150 | 1.5 | 8 | 2425 | 13125 | 17125 |
| 200 | 1.5 | 1.5 | 1797 | 10625 | 14350 |

Even a 5-day treatment with DSG leads to a marked increase and prolongation of antitrypsin expression. The quantitative factor 15 days after discontinuing DSG is approximately 1.8. Continuous treatment (2×weekly) is no longer able to increase this effect dramatically. As a result of the continuous DSG treatment, the expression is only slightly increased by means of the transient administration, but the antibody load against viral proteins is reduced by approximately 90% after 150 days (see Table 2). Even the transient administration of DSG reduces the antibody response in comparison with the control by more than 60% with, at the same time, a markedly positive effect on expression.

TABLE 2
Mean antibody titer

| Days | Control | DSG | DSG++ | FK 506 | Cyclosporin A |
|---|---|---|---|---|---|
| 30 | 54188 | 26310 | 13005 | 65536 | 47923 |
| 100 | 59707 | 31029 | 11251 | 43832 | 32994 |
| 150 | 68555 | 25149 | 7335 | 25149 | 28588 |
| 200 | 45641 | 20586 | 9669 | 17710 | 26021 |

It is interesting that the short-term administration of FK 506 and cyclosporin A is counterproductive in the sense of a long-term expression. Possibly, the immune system reacts with an overshooting activity after discontinuing the immunosuppressant and eliminates the transgenic hepatocytes more rapidly than in the control group.

1. The use of 15-deoxyspergualin, anti-T-cell antibody, corticosteriod, azathioprine, methotrexate or a compound of the formula (I) or (II) 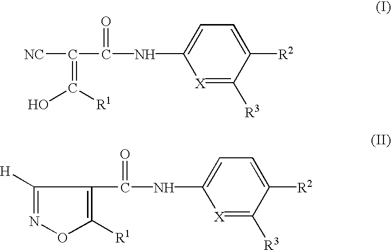
and/or an optionally stereoisomeric form of the compound of the: formula I or II and/or a physiologically tolerable salt of the compound of the formula I, is employed
where
R¹ is
a) (C₁-C₄)-alkyl,
b) (C₃-C₅)-cycloalkyl,
c) (C₂-C₆)-alkenyl or
d) (C₂-C₆)-alkynyl,
R² is
a) —CF₃,
b) —O—CF₃,
c) —S—CF₃,
d) —OH,
e) —NO₂,
f) halogen,
g) benzyl,
h) phenyl,
i) —O-phenyl,
k) —CN or
l) —O-phenyl, mono- or polysubstituted by
1) (C₁-C₄)-alkyl,
2) halogen,
3) —O—CF₃ or
4) —O—CH₃,
R³ is
a) (C₁-C₄)-alkyl,
b) halogen, or
c) a hydrogen atom, and
X is
a) a —CH group or
b) a nitrogen atom,
for the production of a pharmaceutical for increasing the tolerance of a mammal, in particular man, to transgenic cells, after discontinuing the immunosuppressant concomitant therapy, the transgenic cells being transfected by means of a recombinant adenovirus vector.

2. The use as claimed in claim 1, wherein the compound of the formula I and/or II and/or an optionally stereoisomeric form of the compound of the formula I or II and/or a salt of the compound of the formula I is employed, where
R¹ is
a) methyl,
b) cyclopropyl or
c) (C₃-C₅)-alkynyl,
R² is —CF₃ or —CN,
R³ is a hydrogen atom or methyl, and
X is a —CH group.

3. The use as claimed in claim 1 or 2, wherein N-(4-trifluoromethylphenyl)-5-methylisoxazole-4-carboxamide, N-(4-trifluoromethylphenyl)-2-cyano-3-hydroxycrotonamide, 2-cyano-3-cyclopropyl-3-hydroxy acrylic acid (4-cyanophenyl)amide or N-(4-trifluoromethylphenyl)-2-cyano-3-hydroxyhept-2-en-6-ynecarboxamide is employed.

4. The use as claimed in at least one of claims 1 to 3, wherein the pharmaceutical or the pharmaceutical combination is administered before, during and/or after the administration of the transgenic cells produced in vitro or of the in-vivo production of the transgenic cells.

5. The use as claimed in one of claims 1 to 4, wherein the transgenic cells are produced in the course of a gene therapy treatment.

6. The use as claimed in claim 5, wherein the gene therapy treatment is employed for the treatment of all disorders in which a protein or peptide is not produced, is produced inadequately or only produced defectively in the body of the mammal, in particular of man.

7. The use as claimed in claim 5, wherein the gene therapy treatment is employed for the treatment of hereditary disorders such as cystic fibrosis, familial hypercholesterolemia, hemophilia, sickle cell anemia; of nerve and brain disorders such as Parkinson's, Alzheimer's or Kreuzfeld-Jakop syndrome; of rheumatic disorders, osteoarthritis, osteoporosis or arthrosis, of phenylketonuria; of metabolic disorders, such as diabetes; of inflammations; of carcinomatous disorders; of infectious disorders, for example AIDS or hepatitis or of hormone and growth disorders.

8. The use as claimed in claim 5, wherein the gene therapy treatment is employed in order to generate a vaccine protection against disease pathogens such as viruses, bacteria, fungi, mono- and multicellular parasites and also against abnormal body cells such as tumor cells.

9. The use as claimed in at least one of claims 1 to 8, wherein the pharmaceutical or the pharmaceutical combination is administered orally, intravenously, subcutaneously, intraperitoneally, percutaneously, cutaneously, topically, by inhalation, intramuscularly, intrathecally, intraocularly, ocularly, buccally, nasally or rectally, preferably intravenously or orally.
